(19) United States
(12) Patent Application Publication
(10) Pub. No.: US 2015/0050317 A1
(43) Pub. Date: Feb. 19, 2015

(54) Means for Eliciting an Immune Response and a Method Therefor

(71) Applicants: Sonia Moreno-Lopez, Madrid (ES); Marcos Timon-Jimenez, San Lorenzo de El Escorial (ES)

(72) Inventors: Sonia Moreno-Lopez, Madrid (ES); Marcos Timon-Jimenez, San Lorenzo de El Escorial (ES)

(73) Assignee: MOLOGEN AG, Berlin (DE)

(21) Appl. No.: 13/969,862

(22) Filed: Aug. 19, 2013

(51) Int. Cl. A61K 39/29

(52) U.S. Cl. CPC A61K 39/29
USPC 424/196.11

(57) ABSTRACT

A means to elicit a cytotoxic immune response as a result of injecting antigen encoding expression constructs. Minimalistic expression constructs are used that comprise a promoter, a coding sequence and a terminator, and hairpin-shaped oligonucleotides on their ends, to the loop of which cationic peptides are covalently attached. These peptide-coupled expression constructs lead to a cell mediated response type after intradermal injection. The use of a DNA expression construct operable in eucaryotic cells for making a vaccine for intradermal injection to elicit a type 1 cell mediated immune mediated immune response is described.

This application is a Continuation of, and claims priority under 35 U.S.C. §120 to, U.S. patent application Ser. No. 10/816,465, filed Apr. 1, 2004, which was a Continuation-In-Part application of International Patent Application No. PCT/DE02/03798, filed on Oct. 2, 2002, which claims priority from Federal Republic of Germany Patent Application Nos. 101 48 697.9, filed on Oct. 2, 2001, and 101 56 678.6, filed on Nov. 12, 2001. International Patent Application No. PCT/DE02/03798 was pending as of the filing date of this application. The United States was an elected state in International Patent Application No. PCT/DE02/03798.

### BACKGROUND

1. Technical Field

This application concerns the use of a DNA expression construct operable in eucaryotic cells, for the production of a vaccine for intradermal injection to induce a type 1 cellular mediated immune response, as well as a corresponding means for improving the immune response, by linking transfer mediating molecules to gene expression constructs.

This application concerns the field of genetic immunization.

Genetic immunization is based on the principle that gene expression constructs are being inoculated, replacing the traditional method of inoculating attenuated pathogens or their specific antigens. These expression constructs encode immunogenic proteins of viral, bacterial or parasitic pathogens or, in the case of malign pathologies, specifically expressed or presented antigens. The vaccinee is thus only provided with the genetic information for the making of the foreign protein, as a consequence of which somatic cells of the patient produce the foreign protein, and subsequently an efficient immune response against the foreign antigen is being constituted.

2. Background Information

Protection from infectious diseases and also the principle of immunization is based on the recognition by the immune system of structures of pathogens that have been successfully fought against in the past.

Two main pathways are to be distinguished: the humoral pathway, relying on the production of antibodies by B-lymphocytes, but also on humoral components of non-adaptive immunity such as the complement system, and the cellular immune system, which is based on the activity of T-lymphocytes, NK-cells and antigen presenting cells. T-lymphocytes are able to recognize cells that are infected by virus. It is known today that the cellular arm of the immune system is induced by induction of so-called type 1 helper cells, and the humoral arm is induced by activation of so-called type 2 helper cells (Mosmann et al., J. Immuno 1.1986, 136(10): 3561-6). Correspondingly, the cellular arm is also called Th1 pathway and the humoral arm is called Th2 pathway. Bacteria that exist in the extracellular space are usually fought by the Th2 pathway. This pathway is also important for the neutralization of bacterial toxins and the reaction against a diverse set of parasites that can exist in the extracellular space in the body.

Pathogens, on the other hand, that reside mostly in the intracellular space, as it is known for several bacterial species and all viruses, are antagonized mainly by the Th1 pathway, i.e. by cytotoxic cells.

Different methods are known to transfer (“transfect”) the DNA that encodes immunogenic antigens or parts thereof, into the nuclei of antigen presenting cells or other somatic cells, by means of chemical, physical or biological transfection methods.

Means for transfection, so-called “gene shuttles”, are viral vectors, plasmids or covalently closed minimalistic DNA constructs (see EP 0914 318 B1; to be referred to in the following as MIDGE® (MINIMALISTIC IMMUNOLOGICALLY DEFINED GENE EXPRESSION VECTORS)). Wild type virus and vectors closely related thereto generally show a high transfection efficacy and good tissue specificity, but they are deemed controversial due to safety concerns and the problem of anti-vector immunity. This last problem does not exist when using “naked” DNA. When using transfection systems derived from plasmids for in-vivo as well as in-vitro applications, problems arise with regard to efficacy and the specificity of transfection in cell types and tissues. For this reason, attempts have been made to optimize transfection methods that rely on DNA. Different peptides and other organic molecules were linked to gene shuttles by different means of linkage. Also, ligand receptor interactions were utilized by attaching ligands in order to achieve improved uptake of gene shuttles (Fraser et al., 1998, Semin. Immunl., 10 (5): 363-72).

By covalently attaching the nuclear localization signal of the simian virus SV 40 to expression cassettes encoding the hepatitis B virus small surface antigen (HBsAg), a ten to fifteen-fold increased antibody titer could be observed after intramuscular application (Schirmbeck et al., J. Mol Med. 2001 June; 79 (5-6):343-50). A difference was observed with regard to the isotype distribution of the antibody response, with a strong bias towards the Th2-specific IgG1 subtype after intradermal application with particle mediated DNA transfer (“Gene-Gun”), and a bias towards the Th1 specific subtype IgG2a after intramuscular application. No difference was observed that could be correlated to the use of different vectors (plasmid, minimalistic vector).

Similarly, the 11 amino acid T peptide fragment (YGRKKRRQRRR) of the HIV-1 gene product TAT was employed to transfer 200 nm sized liposomes into cells. These experiments however had to use an amount of about 500 T peptides per liposome in order to be successful (Torchilin et al., PNAS 2001, 98(15): 8786-8791). Frankel was able to demonstrate that this peptide comprises a membrane penetrating function that supports the transport of proteins of peptides into cells (Frankel et al., 1988, Cell. 55: 1179-1188, Green et al., 1988, Cell. 55: 1179-1188, U.S. Pat. No. 5,670,617).

It seems of advantage for vaccinating of applying immunotherapy against various diseases to elicit an immune response of the Th1 type. This is particularly true for intracellular parasites such as leishmania and malaria, and viral diseases such as HIV. In this sense, any means of improving the Th1 bias after injection of DNA is advantageous for the formation of an effective response to vaccination.

### Object or Objects

It is the objective of at least one possible embodiment to provide a means to provoke a stronger and more cellular biased (Th1) immune response, as measured per amount of inoculated DNA expression constructs, compared to the means that are provided in the state of the art.

### SUMMARY

This objective is attained in at least one possible embodiment described herein.

It was found that by attaching covalently a peptide comprising a protein transduction domain or a nuclear localization domain, to the hairpin loops of covalently closed DNA constructs (MIDGE), which encode an antigen for vaccination, and subsequent vaccination of the constructs into an animal, that the quality and quantity of the resulting immune response is significantly shifted towards a cellular immune response. In summary, at least one possible embodiment provides a pharmaceutical composition for the provocation of a type 1 cellular mediated immune response by intradermal injection of peptide conjugated DNA expression constructs for the expression of antigens in solution. Among other features, at least one possible embodiment is clearly distinguished from the state of the art, in that exactly the induction of such type 1 response was not expected by the scientific circles involved in the art; as an example for such prejudice, an article by one of the founders of the field of DNA vaccination, David Weiner, in the Journal of Leukocyte Biology (Shedlock and Weiner, J. Leukocyte Biol. Vol 68, December 2000, 793-806) can be cited: the authors explicitly formulate the expectations as to different forms of application (ibid., p. 795 left column below): “Forms of delivery targeting the skin, including i.d. injection . . . have been shown to elicit a humoral response primarily, characterized by a rapid progression to a Th2-type response, associated with the production of an IgA and IgG1 antibody isotype.” A Th1 response was not being expected, which makes at least one of the possible embodiments described herein the more surprising and unexpected for the relevant person skilled in the art.

A cooperation of the assignee with a group at the university of Ulm, Germany (Schirmbeck et al, as cited) was not able to demonstrate changes in antibody level when comparing to unmodified vectors and applying by particle bombardment to the skin. It has to be inferred therefore, that the advantageous effects that are provided according to at least one possible embodiment, are restricted to application by injection into the skin.

This, however, is to be regarded as a great technical improvement. One reason is that the application of DNA expression constructs to the skin is of advantage, since here more of the cells reside that are responsible for the secondary effects of the constructs, and hence less DNA needs to be inoculated. Another reason is that intramuscular injection is more painful and, in farm animals, accompanied by the formation of vaccination scars, which impedes the commercial use of the vaccinated site as meat and contributes to a decrease in value of the vaccinated animal.

The surprising and unexpected result was found that the transducing sequence element of the TAT peptide was able to transport a nucleic acid construct larger by orders of magnitude than its natural substrate. This result was not expected for the reason that so far, experiments had only shown an effect with significantly smaller molecules. Dowdy et al. demonstrated that fusion of this peptide sequence to protein molecules makes it possible to transport a protein of the size of 120 kDalton into cells (Dowdy et al. 2000, Trends Pharmacol. Sci., 21 (2): 45-8). Therefore, we intended to determine whether the TAT derived T peptide is also able to transduce DNA expression constructs that are again much larger. This supposition is not trivial or implicit if only because of the size of the molecule (for an expression construct of 1800 bp this size is 1.2 MDalton) and the completely different shape of the molecule. While proteins and peptides generally are of globular shape, DNA molecules devoid of topological tension can be regarded as linear molecules in first approximation. In considering the differences to proteins, also the surface charge due to the phosphate residues that make DNA a strongly negatively charged molecule, needs to be considered.

Moreover, a peptide sequence was characterized from the simian virus SV 40 that comprises a nuclear localization signal (NLS). The presence of such signal sequences that are necessary for the import of protein into the cellular nucleus, is known from several organisms. Molecules larger than 60 kDa can only be transported into the cellular nucleus by such nuclear localization sequence. In particular, it was demonstrated for the SV-40 NLS that proteins up to 465 kDa can be directed to the nucleus (Lanford et al. 1986, Cell 15; 46 (4): 575-82). This ability of the peptide was utilized here for improving gene transfer. The peptide sequence used is PKKKRKV.

The method to produce such nucleic acid constructs for transcription of RNA molecules in a cell or a complex of cells is based on EP 0 941 318 B1, where the nucleic acid construct

is formed by a circular strand of deoxyribonucleic acid with a base sequence that is partially complementary to the respective other strand and anti-parallel, resulting in a construct shaped like a dumbbell,

where the base sequence that is partially complementary to the respective other strand and anti-parallel, consists mainly of a promoter sequence, a coding sequence and either a polyadenylation signal or another RNA stabilizing sequence element,

and the non-complementary base sequence forms two loops (hairpin loops) comprising single stranded deoxynucleic acid, linking the 5′- and the 3′ end of the base sequence that is partially complementary to the respective other strand and anti-parallel, where

the hairpin loop is formed by at least one of the following oligonucleotides (ODN 1 or ODN 2)

### 1 ODN 1: 5′-PH-GGG AGT CCA GT XT TTC TGG AC ODN 2: 5′-PH-AGG GGT CCA GTT TTC TGG AC,

where X signifies an amino residue modified activated nucleoside residue (thymine),

and an organic molecule is covalently attached to this hairpin loop by means of a crosslinking molecule.

This method enables the covalent attachment of molecules to nucleic acid constructs. In the solutions to the problem known from the state of the art, the attachment of ligands is not achieved by linking the ligands to the plasmid directly, but rather by means of a bridging molecule, and the position of attachment is not defined on the molecular level. Therefore, the danger exists that the function of the promoter or the therapeutic genes is impaired by these modifications. The attachment that is defined on a molecular level also makes it possible to characterize the constructs in accordance to the criteria and regulations applied to modern pharmaceutical production, which is a prerequisite to the use of such vectors as pharmaceuticals. The assignee has found in experiments conducted in house that the effect observed of the induction of a Th-1 response, is not found when peptides are added non-covalently. Covalent attachment is clearly advantageous for the reasons set out above; the reason should be the increased efficacy of transfection and hence, transcription, due to the strong covalent attachment. The effect observed can also be achieved, according to experiments performed by the assignee, by coupling a number of other basic (cationic) peptides, so the scope of the present application is not limited to the use of the two peptide sequences described herein.

In vivo experiments in mice with MIDGE vectors encoding HBsAg and vaccination trials employing the p36 LACK antigen against leishmania major, have corroborated these theoretical considerations. According to at least one possible embodiment, different peptides were attached covalently to the vectors. An efficient and hence safe protection by vaccination was determined by comparing different vaccination regimes. The parameters determined were the strength of the Th2/Th1 shift and the protection attained by vaccination, which was determined by measuring the size of lesions after challenge with leishmania major promastigotes. This is of importance insofar as the leishmania mouse challenge system is a model for the Th1-Th2 dichotomy, and apart from the surrogate parameters that can be measured for every antigen, such as antibody subtype quantity, also the desired biological effect—protection from infection—can be determined in the intact organism. Here, also the great inventive step from using “naked”, not peptide modified DNA to expression constructs attached to cationic peptides, can be observed. While the former confer no significant protection from challenge infection, two applications of peptide modified expression constructs without any further application of interleukins or other immunomodulators resulted in complete protection. This implies a massive deviation of the vaccination regime towards an interferon gamma mediated type 1 immune response (Th1 immune response with interferon gamma cytokine profile and immunoglobulin Th1 subtype bias) by the vaccination regime according to at least one possible embodiment, which could not be detected by surrogate parameters. The vaccine according to at least one possible embodiment is used in solution.

During the presentation of antigen by antigen presenting cells (APC) from naive T-helper cells, a predisposition of the T-helper cells in the direction of either a Th1 (cytotoxic) or a Th2 (humoral) immune response is formed. A determining factor for this predisposition is, among other factors, the cytokine environment in which the interaction between APC and helper cell is taking place, and the nature of the receptors that are taking part in the interaction (Pulendran et al., Science 193, 253-256, 2001).

The isotype distribution of immunoglobulin gamma (IgG) against both antigens (HBsAg and p36/LACK) was determined, since the isotypes IgG1 and IgG2a reflect the bias of the entire immune response. In this regard, IgG1 subtypes are characteristic of a humoral response, accompanied by an increased secretion of interleukins IL-4 and IL-10 by activated lymphocytes; an increased level of subtype IgG2a is typical for a cellular Th1 response, accompanied by increased secretion of IFNg and IL-12. The presence of the subtypes is not exclusive in this context, however the relative titers can be used as an indicator for the dominant type of the immune response that was formed.

Intradermal application of HBsAg encoding plasmids in solution only resulted in a Th2 type immune response (see FIG. 1). A shift towards Th1 type antibody isotypes was not observed (see FIG. 3). A number of medically very important diseases, however, require the formation of a cytotoxic response, among these are the hepatides, leukoviral infections such as HIV and infections by intracellular parasites. In this regard, the formation of a Th1-dominant immune response, which is an objective of at least one possible embodiment, is not only a quantitative improvement, because a higher titer is achieved with less DNA. Rather at least one possible embodiment is a qualitative improvement in comparison to the state of the art, which was not to be expected from the data known from the literature.

The mechanism behind this qualitative shift is not known at present. The fact that ligand attachment to the minimalistic expression vectors leads to an increase in reporter gene expression in vitro (data not shown) does not necessarily predict an improved vaccination, less its Th subtype. A lowering of the amount of applied DNA should, if any prediction could have been made on the basis of the present scientific knowledge, lead to a Th2 response, due to the decrease of the applied immunostimulatory bacterial DNA motifs. With respect to the vectors attached to the NLS and T peptide under investigation here, the opposite is the case.

The results of the vaccination trial against the p36 LACK antigen of L. major show that the means according to at least one possible currently “best” known vaccination regime of secondary immunization (boost) with recombinant vaccinia virus (rVV) that is state of the art. Additionally, it avoids the possible side effects attributed to plasmids and attenuated virus, and is yet comparable in its protective effect (Gonzalo et al., Microbes and Infection: 3 (9):701-711). While of similar or better protective effect, the means according to at least one possible embodiment can be produced more simply, more cheaply and, most importantly, more safely.

The results for the in vivo experiments with HBsAg are very surprising, since they were achieved using a much smaller amount of DNA than was described in the literature so far for the immunization of non-adjuvantated DNA without particulate formulation. For the immunization with HBsAg, generally an amount of 30-100 μg plasmid is used when injecting into muscle or dermis (Schirmbeck et al., 1998, Vaccine. Vol. 16, No. 9/10: 949-954).

The advantages of at least one possible embodiment can be summarized as follows:

minimalistic nucleic acid constructs are modified so as to improve the immune response.

These modified minimalistic nucleic acid constructs lead to an immune response that is more strongly mediated by the cellular arm of the immune system.

The surprising effect of employing the DNA expression construct according to at least one possible embodiment, and a vaccine containing such construct, is exemplified in the presentations contained in the Figures. The abbreviations signify:

2 pMOK p36 plasmid encoding p36 antigen Mp36-NLS MIDGE encoding p36 antigen attached to NLS Peptid pMOK ctr control plasmid encoding HBsAg rVVp36 recombinant Vaccinia Virus encoding p36 phosphate phosphate buffer as control control+ positive control, sera of mice infected with L. major control-negative control, sera of untreated mice

The above-discussed embodiments of the present invention will be described further herein below. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

Further advantageous measures are described herein below; at least one possible embodiment is described in more detail in the following by means of examples and figures. It is shown in

FIG. 1—ELISA determining total IgG directed against Hepatitis small surface Antigen (HBsAg) in mice;

FIG. 2—ELISA determining anti-HBsAg total IgG after boosting with 1 μg DNA;

FIGS. 3 and 4—determination of the anti-HBsAg IgG isotypes IgG 1 and IgG 2a;

FIG. 5—results of a vaccination trial against the p36 LACK antigen of Leishmania major.

FIG. 6—determination of the total IgG antibody titer against p36 LACK antigen after challenge infection with Leishmania major. All vaccination protocols show a measurable antibody response, whereas the highest titer of circulating antibody is provoked by MIDGE p36-NLS/MIDGE p36-NLS.

### DESCRIPTION OF EMBODIMENT OR EMBODIMENTS

### Examples

### 1. Activation of Oligodeoxynucleotides (ODN) for the Attachment of Thiol Functionalised Molecules

Attachment of molecules such as peptides, sugars or other natural compounds can generally be realized by a great number of chemical reactions. These are standard reactions for the formation of amide-, ester- or imide bonds that are sufficiently known from the repertoire of organic synthetic chemistry.

To test the concept, here we employed the reaction of a thiol residue contained in the molecule to be attached, with a maleic acid imide on the nucleic acid molecule. The maleic acid imide residue was introduced into the nucleic acid component by means of the reaction of an amino residue (shown as X) that was introduced during the synthesis of the ODN, with a commercially available bifunctional coupling reagent using a transamidation reaction of a NHS carboxylic acid. For the production of a MIDGE construct modified in one position only, ODN 1 was employed, which comprised in the hairpin loop a desoxy-Uracil (XT) residue modified by an amino residue, and the not modified ODN 2:

5′-PH-GGG AGT CCA GT XT TTC TGG AC (TIB-Molbiol, Berlin, short name: ODN 1=Seq ID 5), where PH signifies 5′-OH phosphorylation, and

5′-PH-AGG GGT CCA GTT TTC TGG AC (TIB-Molbiol, Berlin, short name: ODN 2=Seq ID 6).

The amino modified ODN 1 was employed for attachment as follows: the crosslinking molecule for covalent attachment (here: sulfo-KMUS (N-(Maleimidoundecanoyloxy) sulfosuccinimide) in DMF, PIERCE product-Nr. 21111) was added in four equal parts to the amino-ODN (0.1 mM final concentration) at intervals of 30 min each, until a final concentration of 5 mM was achieved. The reaction took place over two hours in a crosslinking reaction buffer (50 mM NaHPO₄, 75 mM NaCl, pH 7.6) at 37° C. Subsequently, the reaction was stopped by adding of Tris-HCl (pH 7.5; 50 mM final concentration). The activated amino ODN were precipitated for 30 min in −70 C ethanol (300 mM NaOAc pH 5.3; 20 mM MgCl₂; 2.5-fold reaction volume 100% abs. ethanol). The precipitate was centrifuged for 30 min at 15.000 rpm (4 C) and washed under similar conditions 15 min with 70% ethanol. The activated amino ODN were finally dissolved in water (MilliQ quality) and stored at −20 C until further use.

### 2. Attachment of T Peptide to Activated Oligodeoxynucleotides (ODN)

The activated amino ODN described under 1) was dissolved in coupling reaction buffer (1.times.=50 mM NaHPO₄, 75 mM NaCl, pH 7.0) to achieve a final concentration of 0.1 mM. Subsequently, the T peptide, dissolved in water, comprising the sequence YGRKKRRQRRR (=Seq ID 3; produced and provided by Dr. Peter Henklein, Charit, Berlin) was added at 0.2 mM final concentration. The reaction was allowed to continue for 1 hour at 37° C.

Purification and separation of the resulting peptide-attached ODN from not reacted ODN was achieved by reversed phase HPLC. Single reaction batches were analysed by gel electrophoresis. The fraction containing the T-ODN was concentrated in a vacuum centrifuge and dissolved in ultra pure water. The modified ODN were purified employing a Nukleosil-300 C18 column (10 μm, 250 mm length×8 mm bore) by HPLC. The gradient continued from 0% buffer A (100 mM ammonium carbonate) to 42% buffer B (80% acetonitrile) over 47 min at a flow rate of 2.4 ml/min.

### 3. Coupling of SV-40 NLS Peptide to Activated ODN

The activated amino ODN described under 1) was dissolved in coupling reaction buffer (1.times.=50 mM NaHPO₄, 75 mM NaCl, pH 7.0) to achieve a final concentration of 0.1 mM. Subsequently, the SV-40 NLS peptide, dissolved in water, comprising the sequence PKKKRKV (=Seq ID 4; Dr. Henklein) was added at 0.2 mM final concentration. The reaction was allowed to continue for 1 hour at 37° C.

Purification and separation of the resulting peptide attached ODN from not reacted ODN was achieved by reversed phase HPLC. Single reaction batches were analysed by gel electrophoresis. The fraction containing the NLS-ODN was concentrated in a vacuum centrifuge and dissolved in ultra pure water.

### 4. Production of MIDGE-HBsAg T-Peptide

MIDGE are minimalistic expression vectors made of double stranded DNA that only consist of the expression cassette, that is the CMV promoter, an intron, the respective gene sequence and a polyadenylation sequence. The constructs were obtained as follows: the plasmid pMOK HBsAg was digested to completion by Eco31I. Ligation with 5′ phosphorylated hairpin-shaped ODN 1, to which the T peptide was attached according to example 1, and ODN 2, was achieved using T4 DNA ligase in the presence of Eco31I, and stopped by heating to 70° C. The resulting mix was concentrated and treated with Eco31I and T7 DNA polymerase in the absence of deoxyribonucleotide trisphosphates. Purification was achieved by anion exchange chromatography. Verification of the successful coupling of the different ligands was performed by restriction enzyme digestion. By comparing, by means of gel electrophoresis, the sizes of ODN digested (with BamHI or Eco31I) from the MIDGE vector to the respective control ODN, it could be shown that the vectors were linked to peptides. (The sequence HBsAg is represented in Seq. ID 1).

The production of MIDGE HBsAg NLS-peptide was performed analogously.

### 5. Recombinant Construction of the Plasmid pMOKp36

2 fragments were amplified by PCR from the starter plasmid pSCp36:

3 1. PCR approx. 800 bp;
Primer: left
(= Seq ID 7)
5′-TTATATGGTACCATGAACATACGAGGGTCACCT,
Primer: right
(= Seq ID 8)
5′-TTATATGAGCTCAGAAGACACGGACAGGGACCTCTTCCGTCG
2. PCR approx. 950 bp;
Primer: left
(= Seq ID 9)
5′-TTATATGGTACCATGAACATACGAGGGTCACCT,
Primer: right
(= Seq ID 10)
5′-TTATATGAGCTCTTACTCGGCCGTCGGAGATGG

The PCR product derived from the second PCR reaction was digested by Eco31I and the smaller fragment (approx. 200 bp) was isolated.

The PCR product from the first PCR reaction was digested with Bpil.

The 200 bp fragment and the digested fragment from the first PCR reaction were ligated and subsequently digested by KpnI and SacI, and inserted by ligation into the pMOK vector that had been digested by KpnI and SacI. The resulting plasmid was named pMOK p36. It was used to produce MIDGE p36-NLS (The sequence p36 LACK is represented in Seq. ID 2).

Activation of ODN, attachment of the NLS sequence to the oligonucleotides and the production of the MIDGE p36-NLS was performed as described above.

Results

The results obtained are described in detail in the following figures:

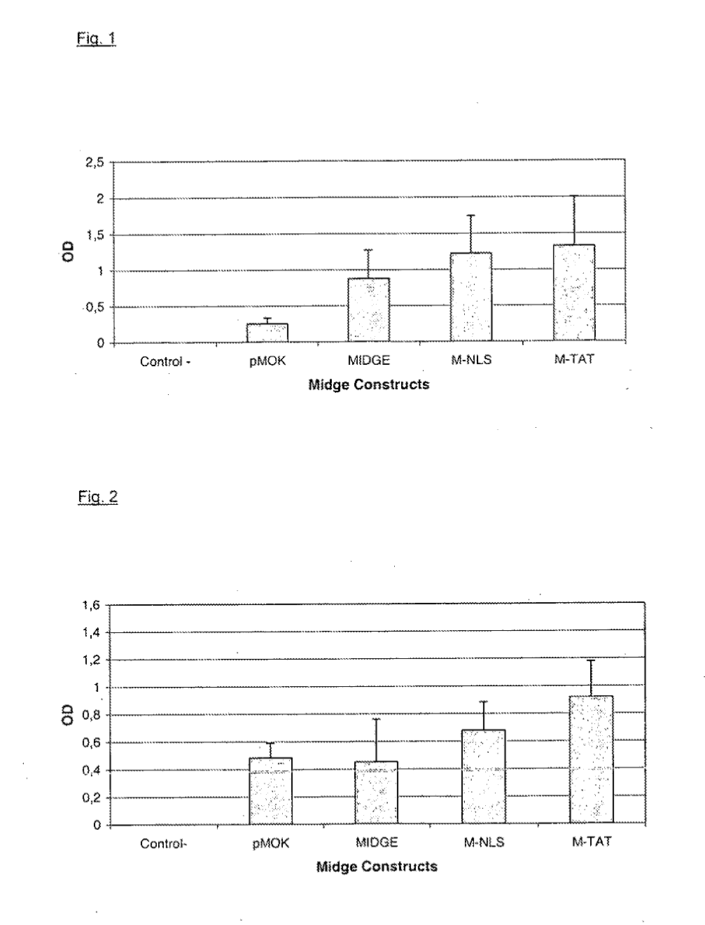

FIG. 1 shows the determination of total IgG HBsAg level. Antibody levels were determined by ELISA, and the absorption was read as OD (optical density) at a wavelength of λ=450 nm. As a comparison, plasmid and unmodified MIDGE vector was employed. MIDGE with different attachments showed a clear increase of the antibody titre, which indicates an increased expression of the HBsAg. The abbreviations are used:

4 pMOK: plasmid. MIDGE: unmodified MIDGE M-NLS: MIDGE attached to NLS M-TAT: MIDGE attached to T peptide

FIG. 2 shows the booster effect of a secondary immunization with 1 μg DNA after 11 weeks. The amount of DNA immunized with in the primary and the secondary immunization was 1 μg DNA. The effect of modified MIDGE was again a significant increase of the immune response in this experiment.

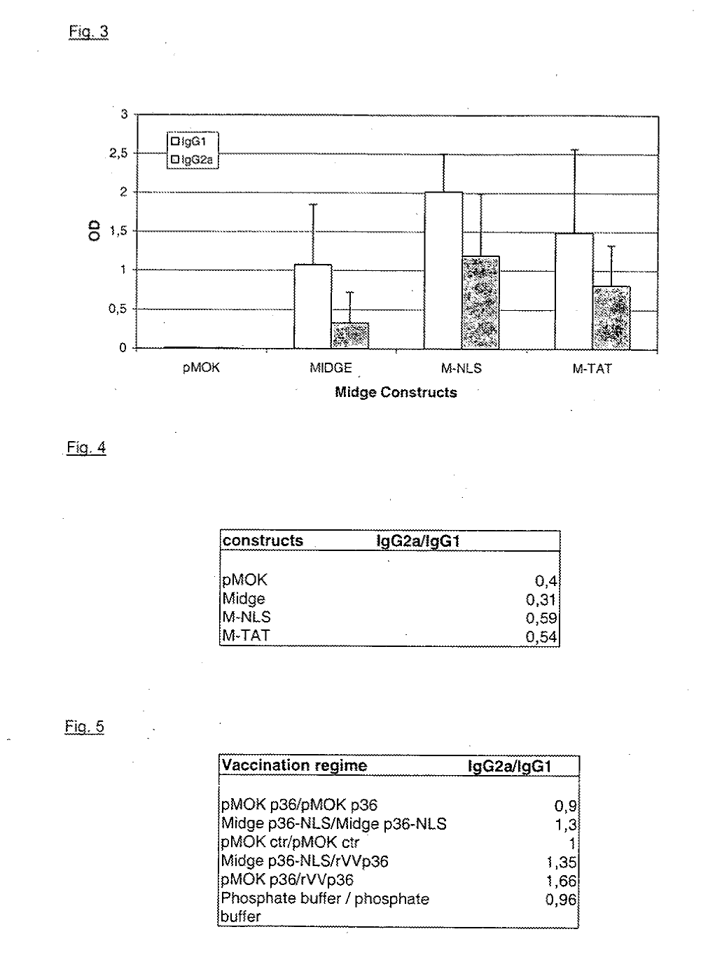

FIGS. 3 and 4 show the determination of IgG isotypes IgG 1 and IgG 2a to HBsAg. Surprisingly it was found that MIDGE coupled to the T peptide and to the NLS sequence elicited a cytotoxic immune response (Th1), as indicated by the antibody isotype distribution.

FIG. 5 shows the ratio of the antibody isotype distribution IgG 2a and IgG 1 after secondary immunization and challenge infection with leishmania major promastigotes. The immunization regime MIDGE p36-NLS/MIDGE p36-NLS shows the unexpected effect of eliciting a cellular (Th1) immune response. The Th2/Th1 shift in the immune response elicited by the regime pMOKp36/rVVp36 is only marginally different in comparison.

### Experimental Example 1

### Determination of HBsAg Antibody in Mice

According to example 4, MIDGE encoding the hepatitis B surface antigen (subtype ay) were produced. Proof of the expression of the encoded antigen was performed by antibody titre determination against hepatitis B antigen by means of ELISA. The production of MIDGE was performed according to example 4. In particular, unmodified MIDGE and MIDGE with a ligand, specifically MIDGE-NLS and MIDGE-T, were produced. As an additional control, the plasmid pMOK HBsAg was used. As a negative control the sera of untreated mice were used.

MIDGE (unmodified as well as NLS-modified) and plasmid were dissolved in sodium phosphate pH 7.2 in a volume of 50 μl and injected into Balb/c mice intradermally. DNA amounts used were 10 μg and 1 μg per animal and per vaccination, respectively. 5 animals were used per group. After 11 weeks, a secondary immunization (boost) was performed. Determination of antibody from sera was performed at week 2, 4 and 8. The results are shown in FIG. 1. When using 10 μg DNA, a clear increase of total immunoglobulin G (IgG) titre is seen in week 4, indicating an increased expression of HBsAg by all MIDGE constructs in comparison to plasmid. The greatest effect was elicited by modified MIDGE. The error bars show the standard deviation. Using 1 μg DNA primarily did not lead to a significant increase of the HBsAg titre in week 4 (results not shown), however a surprising strong increase of the titre was found after the boost at week 11. Again, modified MIDGE showed the strongest effect (see FIG. 2). These results show that even with a minimal amount of DNA a high antibody titre is attained with MIDGE.

### Example 2

### Vaccination Trial Against the Leishmania p36 Antigen

In order to elicit an effective protection by vaccination against leishmania major, the 36 kDa antigen, also referred to as LACK, was used. In the vaccination trial, different gene shuttles were employed that all encoded the immunogenic p36 antigen: MIDGE with NLS attachment, plasmid pMOKp36 and recombinant vaccinia virus p36 (rVV). In order to obtain a comparison to the most effective prime/boost vaccination, constructs were injected into female mice (Balb/c) according to the following scheme:

5 primary Secondary immunization group immunization. (boost) 1 pMOKp36 pMOKp36 2 MIDGE p36-NLS MIDGE p36-NLS 3 pMOK control pMOK control 4 pMOK p36 rVVp36 5 MIDGE p36-NLS rVVp36 7 phosphate buffer phosphate buffer

10 mice were used per group.

Amounts of DNA were:

pMOK p36: 100 μg, i.d.

MIDGE p36-NLS: 54.8 μg, i.d.

rVV p36: 5×10⁷ pfu/animal, i.p.

and were applied dissolved in sodium phosphate buffer at pH 7.2.

After 2 weeks, the secondary immunization (boost) was performed with the respective DNA construct (see scheme). Three weeks after the boost, challenge infection was performed with 5×10⁴ leishmania major promastigotes. These were injected into the right hind paw subcutaneously.

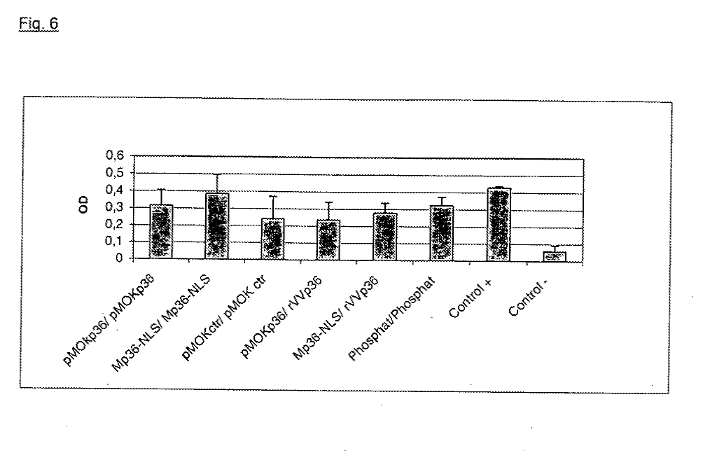

Eight weeks after the challenge infection, all mice were bled for sera (see FIG. 6). Determination of total IgG antibody titre against p36 and the determination of IgG 2a and IgG 1 was performed by means of ELISA, reading absorption as optical density at a wavelength of λ=406 nm.

One feature or aspect of an embodiment is believed at the time of the filing of this patent application to possibly reside broadly in a use of a DNA expression construct operable in eucaryotic cells for the production of a vaccine for intradermal injection for eliciting of a type 1 cellular mediated immune response, where said DNA expression construct is a covalently closed linear deoxyribonucleotide molecule comprising a linear double stranded region, where the single strands forming the double strand are linked by a short single stranded loop consisting of deoxyribonucleotides, where said double strand forming single strands only consist of the coding sequence under control of a promoter that is operable in the animal that is to be vaccinated, and a terminator sequence and the DNA expression construct is linked covalently to one or more oligopeptides to increase transfection efficacy.

Another feature or aspect of an embodiment is believed at the time of the filing of this patent application to possibly reside broadly in the use of the DNA expression construct, where said construct encodes the hepatitis small surface antigen (HBsAg).

Yet another feature or aspect of an embodiment is believed at the time of the filing of this patent application to possibly reside broadly in the use of the DNA expression construct, where the oligopeptide is of a length of five to 25 amino acids and at least half of the amino acids are a member of the group consisting of lysine and arginine.

Still another feature or aspect of an embodiment is believed at the time of the filing of this patent application to possibly reside broadly in the use of the DNA expression construct, where the oligopeptide comprises a nuclear localisation sequence.

A further feature or aspect of an embodiment is believed at the time of the filing of this patent application to possibly reside broadly in the use of the DNA expression construct, where the oligopeptide comprises the sequence PKKKRKV (proline-lysine-lysine-lysine-arginine-lys-ine-valine).

Another feature or aspect of an embodiment is believed at the time of the filing of this patent application to possibly reside broadly in the use of the DNA expression construct, where the oligopeptide comprises the sequence YGRKKRRQRRR.

Yet another feature or aspect of an embodiment is believed at the time of the filing of this patent application to possibly reside broadly in a vaccine for intradermal injection to elicit a type 1 cellular mediated immune response employing the DNA expression construct.

Still another feature or aspect of an embodiment is believed at the time of the filing of this patent application to possibly reside broadly in the vaccine, where the vaccine is present in solution.

A further feature or aspect of an embodiment is believed at the time of the filing of this patent application to possibly reside broadly in the vaccine for the application in human beings.

Another feature or aspect of an embodiment is believed at the time of the filing of this patent application to possibly reside broadly in a use of a DNA expression construct operable in eucaryotic cells for the production of a vaccine for intradermal injection for eliciting of a type 1 cellular mediated immune response, where said construct encodes one or more antigens under control of a promoter sequence and the DNA expression construct is linked covalently to one or more oligopeptides to increase transfection efficacy.

Yet another feature or aspect of an embodiment is believed at the time of the filing of this patent application to possibly reside broadly in the use of the DNA expression construct, where the immunizing polynucleotide sequences are present in the form of expression constructs that consist of covalently closed linear deoxyribonucleic acid molecules, which comprise a linear double stranded region, where the single strands forming the double strand are linked by a short single stranded loop consisting of deoxyribonucleotides, where said double strand forming single strands only consist of the coding sequence under control of a promoter that is operable in the animal that is to be vaccinated, and a terminator sequence.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

Some examples of universal primers which may possibly be utilized in at least one possible embodiment may possibly be found in the following U.S. Pat. Nos. 6,207,372 and 5,882,856.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

Some examples of methods of and devices for performing a polymerase chain reaction (PCR) which may possibly be utilized in at least one possible embodiment may possibly be found in the following U.S. Pat. Nos. 6,596,492; 6,586,250; 6,586,233; 6,569,678; 6,569,627; 6,566,067; 6,566,052; 6,558,929; 6,558,909; 6,551,783; 6,544,782; 6,524,830; 6,518,020; 6,514,750; 6,514,706; 6,503,750; 6,493,640; 6,492,114; 6,485,907; and 6,485,903.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

Some examples of restriction enzymes which may possibly be utilized in at least one possible embodiment may possibly be found in the following U.S. Pat. Nos. 6,495,325; 6,403,354; 6,258,539; 6,015,663; 5,955,369; 5,789,226; 5,470,732; 5,250,429; 5,179,016; 5,175,101; 5,165,933; 5,120,651; 4,960,707; 4,833,082; 4,808,531; 4,724,209; 4,668,631; and 4,542,099.

All of the patents, patent applications and publications recited herein, and in the Declaration attached hereto, are hereby incorporated by reference as if set forth in their entirety herein.

Some examples of methods of and devices for performing restriction digestion which may possibly be utilized in at least one possible embodiment may possibly be found in the following U.S. Pat. Nos. 6,506,568; 6,046,039; 5,879,950; 5,595,870; and 3,953,609.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

Some examples of the restriction enzyme Eco31I which may possibly be utilized in at least one possible embodiment may possibly be found in the following U.S. Pat. Nos. 6,599,703; 6,579,705; 6,451,563; 6,344,345; 6,303,308; 6,258,533; 6,190,889; 5,858,671; 5,658,736; 5,468,851; 5,436,150; 5,356,802; and 5,278,051.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

Some examples of methods of and devices for performing gel electrophoresis which may possibly be utilized in at least one possible embodiment may possibly be found in the following U.S. Pat. Nos. 6,582,574; 6,576,104; 6,569,306; 6,535,624; 6,406,602; 6,379,515; 6,301,377; 6,258,544; 6,197,173; 6,190,522; 6,127,134; 6,057,106; 6,043,025; 6,001,233; 5,989,400; 5,972,188; 5,938,909; 5,938,906; 5,916,427; and 5,904,826.

All of the patents, patent applications or patent publications, which were cited in the international search report dated Sep. 11, 2003, and/or cited elsewhere are hereby incorporated by reference as if set forth in their entirety herein as follows: SCHIRMBECK REINHOLD ET AL, “Priming of immune responses to hepatitis B surface antigen with minimal DNA expression constructs modified with a nuclear localization signal peptide,” JOURNAL OF MOLECULAR MEDICINE (BERLIN), Bd. 79, Nr. 5-6, Juni 2001 (2001-06); MCCLUSKIE M J ET AL, “ROUTE AND METHOD OF DELIVERY OF DNA VACCINE INFLUENCE IMMUNE RESPONSES IN MICE AND NON-HUMAN PRIMATES”, MOLECULAR MEDICINE, BLACKWELL SCIENCE, CAMBRIDGE, MA, US, Bd. 5, Nr. 5, Mai 1999 (1999-05); SHI NING ET AL, “Immune responses affected by different injection methods of a multi-epitope chimeric DNA vaccine of Plasmodium falciparum,” ZHONGHUA WEISHENGWUXUE HE MIANYIXUE ZAZHI, Bd. 21, Nr. 1, January 2001 (2001-01); EP 0 941 318 A (SOFT GENE GMBH) 15. September 1999 (1999 Sep. 15); LOPEZ-FUERTES L ET AL, “DNA vaccination with linear minimalistic (MIDGE) vectors confers protection against Leishmania major infection in mice” VACCINE, BUTTERWORTH SCIENTIFIC. GUILDFORD, GB, Bd. 21, Nr. 3-4, 13. December 2002 (2002 Dec. 13); and “Form Follows Function: Introduction to the MIDGE Vector Technology,” INTERNET PUBLICATION, ‘Online! 31. Mai 2002 (2002 May 31), XP002252259 Gefunden im Internet: <URL:http://web.archive.org/web/2002060214 1807/www.midge.com/technology/index.html>.

Some examples of methods of and devices for performing DNA injection which may possibly be utilized in at least one possible embodiment may possibly be found in the following U.S. Pat. Nos. 6,319,224; 6,294,064; 6,214,804; 5,656,610; and 5,589,466.

The corresponding foreign and international patent publication applications, namely, Federal Republic of Germany Patent Application No. 101 48 697.9, filed on Oct. 2, 2001, having inventors Sonia MORENO-LPEZ and Marcos TIMN-JIMENZ, and DE-OS 101 48 697.9 and DE-PS 101 48 697.9, and Federal Republic of Germany Patent Application No. 101 56 678.6, filed on Nov. 12, 2001, having inventors Sonia MORENO-LPEZ and Marcos TIMN-JIMENZ, and DE-OS 101 56 678.6 and DE-PS 101 56 678.6, and International Application No. PCT/DE02/03798, filed on Oct. 2, 2002, having WIPO Publication No. WO03/031469 and inventors Sonia MORENO-LPEZ and Marcos TIMN-JIMENZ, as well as their published equivalents, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

Some examples of methods of and devices for performing gene or genetic injection which may possibly be utilized in at least one possible embodiment may possibly be found in the following U.S. Pat. Nos. 6,525,030; 6,361,991; 6,090,790; 5,998,382; 5,697,901; 5,661,133; 5,273,525; 6,482,405; and 6,063,629.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The following U.S. patent applications are hereby incorporated by reference as if set forth in their entirety herein: Ser. No. 10/057,311, filed Jan. 24, 2002, entitled “Covalently Closed Nucleic Acid Molecules for Immunostimulation,” and having inventors Junghans, et al. and attorney docket no. NHL-NP-37; Ser. No. 10/041,672, filed Jan. 8, 2002, entitled “Feline Interleukin-12 as Immunostimulant,” and having inventors Lutz, et al. and attorney docket no. NHL-NP-36; and Ser. No. 10/816,591, filed Apr. 1, 2004, entitled “DNA Expression Construct for Treatment of Infections with Leishmaniasis,” and having inventors Laura FUERTES-LPEZ and Marcos TIMN-JIMENZ and attorney docket no. NHL-NP-46,

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The following U.S. patents may possibly disclose structures or processes that may possibly be used in at least one possible embodiment of the present invention, and are hereby incorporated by reference as follows: U.S. Pat. No. 6,534,271, issued to Furste, et al. on Mar. 18, 2003; U.S. Pat. No. 6,451,593 issued to Wittig, et al. on Sep. 17, 2002; and U.S. Pat. No. 6,451,563 issued to Wittig, et al. on Sep. 17, 2002.

The details in the patents, patent applications and publications may be considered to be incorporable, at applicant's option, into the claims during prosecution as further limitations in the claims to patentably distinguish any amended claims from any applied prior art.

The following U.S. patents and foreign patent publications may possibly disclose structures or processes that may possibly be used in at least one possible embodiment of the present invention, as follows: U.S. Pat. No. 5,580,859; U.S. Pat. No. 5,584,807; U.S. Pat. No. 5,589,466; DE 198 54 946; DE 196 48 625; DE 198 26 758; EP 0686697; EP 0732395; WO 9626270; WO 9632473; WO 92/13963; WO 9313216; WO 94/12633; and WO 98/21322.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The following foreign patent publications are hereby incorporated by reference as if set forth in their entirety herein: International Application No. PCT/DE02/03799 filed Oct. 2, 2002 and corresponding publication WO03/031470 published Apr. 17, 2003, Federal Republic of Germany Patent Application No. 101 48 732.0 filed Oct. 2, 2001, and Federal Republic of Germany Patent Application No. 101 56 679.4 filed Nov. 12, 2001.

The abstract of the disclosure is submitted herewith as required by 37 C.F.R. .sctn.1.72(b). As stated in 37 C.F.R. .sctn.1.72(b):

A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, any statements made relating to the abstract are not intended to limit the claims in any manner and should not be interpreted as limiting the claims in any manner.

The following publications discuss genetic technology, such as gene therapy, DNA cloning, production, and manipulation thereof, and treatment and immunization of cells with DNA, and may possibly disclose structures or processes that may possibly be used in at least one possible embodiment of the present invention. These publications are incorporated by reference as follows: Eck, et al., 1996. Goodman & Gilman's The Pharmacological Basis of Therapeutics, Ninth Edition. McGraw-Hill, New York; Johnston, et al., 1993. Genetic Engineering, 15:225-236; “Immunization by Direct DNA Inoculation Induces Rejection of Tumor Cell Challenge” Wang et al., Human Gene Therapy 6:407-418 (April 1995); “Identification of Wild-Type and Mutant p53 Peptides Binding to HLA-A2 Assessed by a Peptide Loading-Deficient Cell Line Assay and Novel Major Histocompatibilty Complex Class I Peptide Binding Assay” Stuber et al., Eur. J. Immunol. 1994. 24:765-768; “Particle-Mediated Gene Transfer of Granulocyte-Macrophage Colony-Stimulating Factor cDNA to Tumor Cells: Implications for a Clinically Relevant Tumor Vaccine” Mahvi et al., Human Gene Therapy 7:1535-1543 (Aug. 20, 1996); “Ex Vivo Regulation of Specific Gene Expression by Nanomolar Concentration of Double-Stranded Dumbbell Oligonucleotides” Clusel et al., Nucleic Acids Research, 1993, vol. 21, No. 15, 3405-3411; “Dendritic Cells as Initiators of Tumor Immune Responses: A Possible Strategy for Tumor Immunotherapy?” Grabbe et al., Immunology Today, vol. 16, No. 3 1995, 117-121; “Sequence-independent Inhibition of RNA Transcription by DNA Dumbbells and Other Decoys” Lim et al., Nucleic Acids Research, 1997, vol. 25, No. 3, 575-581; “A New Peptide Vector for Efficient Delivery of Oligonucleotides into Mammalian Cells” Morris et al., Nucleic Acids Research, 1997, vol. 25, No. 14, 2730-2736; “Improved Biological Activity of Antisense Oligonucleotides Conjugated to a Fusogenic Peptide” Bongartz et al., Nucleic Acids Research, 1994, vol. 22, No. 22, 4681-4688; “The Influence of Endosome-Disruptive Peptides on Gene Transfer Using Synthetic Virus-Like Gene Transfer Systems” Plank et al., The Journal of Biological Chemistry, vol. 269, No. 17, Apr. 29, pp. 12918-12924, 1994; “Linear Mitochondrial DNAs of Yeasts: Closed-Loop Structure of the Termini and Possible Linear-Circular Conversion Mechanisms” Dinouel et al., Molecular and Cellular Biology, April 1993, pp. 2315-2323; “Heterologous Protection Against Influenza by Injection of DNA Encoding a Viral Protein” Ulmer et al., Science, vol. 259, Mar. 19, 1993, pp. 1745-1749; “Comparison of Organic Monolayers on Polycrystalline Gold Spontaneously Assembled from Solutions Containing Dialkyl Disulfides or Alkanethiols” Biebuyck et al., Langmuir 1994, 10, 1825-1831; “Regression of Established Murine Carcinoma Metastases Following Vaccination with Tumour-Associated Antigen Peptides” Mandelboim et al., Nature Medicine, vol. 1, No. 11, November 1995, pp. 1179-1183; Kilisch et al. Covalently linked sequencing primer linkers (stinkers) for sequence analysis of restriction fragments. Gene vol. 44, pp. 263-270, December 1986; Roberts, R J. Restricition and modification enzymes and their recognition sequences. vol. 13 Suppl. r165-r200, December 1985; and Berger and Kimmel. Guide to molecular cloning techniques. Methods in Enzymology. vol. 52, Academic Press, Inc. New York. pp. 307-661, December 1987.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

We claim:

1. A method of vaccinating a living being to protect against infectious diseases caused by infectious agents or germs using a vaccine comprising a DNA expression; said method comprising the steps of:
A) injecting the vaccine comprising a DNA expression construct comprising a covalently-closed, linear, dumbbell-shaped deoxyribonucleic acid molecule; said deoxyribonucleic acid molecule comprising a linear double-stranded region; said double-stranded region comprising single strands being linked by a short, single-stranded loop consisting of deoxyribonucleic acid nucleotides; said double-strand-forming single strands comprising: a terminator sequence, and a coding sequence for one or more antigens under the control of a promoter that is configured to be operable in the living being that is to be vaccinated; and said DNA expression construct is linked covalently to at least one oligopeptide to increase transfection efficacy into the living being; and
B) eliciting, with said DNA expression construct of said injected vaccine, a type 1 cellular mediated immune response in the living being against intracellular infection germs that cause infectious diseases.

2. The method of vaccinating of claim 1, wherein said step of injecting said vaccine comprising said DNA expression construct into a living being comprises intradermally injecting said vaccine.

3. The method of vaccinating of claim 1, wherein said DNA construct encodes the hepatitis small surface antigen (HBsAg).

4. The method of vaccinating of claim 1, wherein the oligopeptide is of a length of five to 25 amino acids and at least half of the amino acids are selected from the group consisting of lysine and arginine.

5. The method of vaccinating of claim 1, wherein the oligopeptide comprises a nuclear localization sequence.

6. The method of vaccinating of claim 1, wherein the oligopeptide comprises the sequence PKKKRKV (SEQ ID NO. 4).

7. The method of vaccinating of claim 1, wherein the oligopeptide comprises the sequence YGRKKRRQRRR (SEQ ID NO. 3).

8. The method of vaccinating of claim 1, wherein said step of injecting said vaccine comprising said DNA expression construct into a living being comprises injecting said vaccine comprising said DNA expression construct into an animal or a human being.

9. The method of claim 8, wherein said step of injecting into an animal or a human being comprises intradermally injecting said vaccine.

10. The method of claim 1, wherein intracellular infection germs cause said infectious disease.

11. A vaccine comprising:
A) a type 1-cellular-mediated-immune-response-eliciting vaccine being configured to be injected into a living being to protect against infectious diseases caused by infectious agents or germs; said type 1-cellular-mediated-immune-response-eliciting vaccine comprising:
i) a DNA expression construct being configured to operate in eukaryotic cells; said DNA expression construct comprising a covalently-closed, linear, dumbbell-shaped deoxyribonucleic acid molecule; said deoxyribonucleic acid molecule comprising a linear double-stranded region; said double-stranded region comprising single strands being linked by a short, single-stranded loop consisting of deoxyribonucleic acid nucleotides; said double-strand-forming single strands comprising: a terminator sequence, and
ii) a coding sequence for one or more antigens under the control of a promoter that is configured to be operable in the living being that is to be vaccinated;
B) at least one oligopeptide being linked covalently to said DNA expression construct said at least one oligopeptide to increase transfection efficacy.

12. The vaccine of claim 11, wherein said vaccine is configured to be injected intradermally.

13. The vaccine of claim 11, wherein said vaccine is configured to be injected in a solution.

14. The vaccine of claim 11, wherein said vaccine is configured to be injected in human beings.

15. A method of vaccinating a living being to protect against infectious diseases caused by infectious agents or germs using a vaccine comprising a DNA expression; said method comprising the steps of:
A) injecting said vaccine comprising a DNA expression construct comprising a covalently-closed, linear, dumbbell-shaped deoxyribonucleic acid molecule; said deoxyribonucleic acid molecule comprising a linear double-stranded region; said double-stranded region comprising single strands being linked by a short, single-stranded loop comprising deoxyribonucleic acid nucleotides; said double-strand-forming single strands comprising: a terminator sequence, and a coding sequence under the control of a promoter that is configured to be operable in the living being that is to be vaccinated; and said DNA expression construct is linked covalently to at least one oligopeptide to increase transfection efficacy into a living being; and
B) eliciting, with said DNA expression construct of said injected vaccine, a type 1 cellular mediated immune response in the living being against intracellular infection germs that cause infectious diseases.

16. The method of vaccinating of claim 15, wherein said step of injecting said vaccine comprising said DNA expression construct into a living being comprises intradermally injecting said vaccine.

17. The method of vaccinating of claim 15, wherein said construct encodes the hepatitis small surface antigen (HBsAg).

18. The method of vaccinating of claim 15, wherein the oligopeptide is of a length of five to 25 amino acids and at least half of the amino acids are selected from the group consisting of lysine and arginine.

19. The method of vaccinating of claim 15, wherein the oligopeptide comprises a nuclear localization sequence.

20. The method of vaccinating of claim 15, wherein the oligopeptide comprises the sequence PKKKRKV (SEQ ID NO. 4).

21. The method of vaccinating of claim 15, wherein the oligopeptide comprises the sequence YGRKKRRQRRR (SEQ ID NO. 3).

22. The method of vaccinating of claim 15, wherein said step of injecting said vaccine comprising said DNA expression construct into a living being comprises injecting said vaccine comprising said DNA expression construct into an animal or a human being.

23. The method of claim 22, wherein said step of injecting into an animal or a human being comprises intradermally injecting said vaccine.

24. The method of claim 15, wherein intracellular infection germs cause said infectious disease.


SEQUENCE LISTING (10 sequences)

SEQ ID NO: 1 - DNA, 4533 bp, artificial sequence
  pMOK HBsAg
tcttccgcttcctcgctcactgactcgctgcgctcggtcgttcggctgcggcgagcggtatcagctcactcaaaggcggtaatacggttatccacagaatcaggggataacgcaggaaagaacatgtctcgggaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggtctcctcccggatcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagaagctttattgcggtagtttatcacagttaaattgctaacgcagtcagtgctcgagcaggtaagtatcaaggttacaagacaggtttaaggaggccaatagaaactgggcttgtcgagacagagaagactcttgcgtttctgataggcacctattggtcttactgacatccactttgcctttctctccacaggggtaccatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctctgggtatacatttaagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcagggggagacccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 2 - DNA, 4791 bp, artificial sequence
  Plasmid pSCp36
tcttccgcttcctcgctcactgactcgctgcgctcggtcgttcggctgcggcgagcggtatcagctcactcaaaggcggtaatacggttatccacagaatcaggggataacgcaggaaagaacatgtctcgggaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggtctcctcccggatcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagaagctttattgcggtagtttatcacagttaaattgctaacgcagtcagtgctcgagcaggtaagtatcaaggttacaagacaggtttaaggaggccaatagaaactgggcttgtcgagacagagaagactcttgcgtttctgataggcacctattggtcttactgacatccactttgcctttctctccacaggggtaccatgaactacgagggtcacctgaagggccaccgcggatgggtcacctccctggcctgcccgcagcaggcggggtcgtacatcaaggtggtgtcgacgtcgcgcgatggcacggccatctcgtggaaagccaaccccgaccgccacagcgtggacagcgactacggtctgccgagccaccgcctcgagggccacaccggcttcgtgtcgtgtgtgtcgctggcccacgccaccgactacgcgctgaccgcgtcctgggaccgctccatccgcatgtgggacctgcgcaatggccagtgccagcgcaagttcctgaagcacaccaaggacgtgctcgccgtcgccttctcgccggacgaccgcctgatcgtgtccgcgggccgcgacaacgtgatccgcgtgtggaacgtggcgggcgagtgcatgcacgagttcctgcgcgacggccacgaggactgggtgagcagcatctgtttctcgccgtcgctggagcatccgatcgtggtgtccggcagctgggacaacaccatcaaggtatggaacgtgaacgggggcaagtgtgagcgcacgctcaagggccacagcaactacgtgtccacggtgacggtgtcgccagacgggtcgctgtgcgcgtccggcggcaaggacggcgcggcgctgctgtgggacctgagcaccggcgagcagctgttcaagatcaacgtggagtcgcccatcaaccagatcgccttctcgcccaaccgcttctggatgtgcgtcgcgacggagaggtccctgtccgtgtacgacctggagagcaaggctgtgattgcggagctgacgccggacggcgcgaagccgtccgagtgcatctccattgcctggtccgccgacggcaacactctgtactccggtcacaaggacaacctgatccgcgtgtggtccatctccgacgccgagtaagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcagggggagacccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 3 - protein, 11 aa, Human immunodeficiency virus type 1
  MISC_FEATURE: TAT peptide
YGRKKRRQRRR

SEQ ID NO: 4 - protein, 7 aa, Simian virus 40
  MISC_FEATURE: NLS peptide
PKKKRKV

SEQ ID NO: 5 - DNA, 20 bp, artificial sequence
  ODN 1
gggagtccagttttctggac

SEQ ID NO: 6 - DNA, 20 bp, artificial sequence
  ODN 2
aggggtccagttttctggac

SEQ ID NO: 7 - DNA, 33 bp, artificial sequence
  1. PCR Primer left
ttatatggtaccatgaacatacgagggtcacct

SEQ ID NO: 8 - DNA, 42 bp, artificial sequence
  1. PCR Primer right
ttatatgagctcagaagacacggacagggacctcttccgtcg

SEQ ID NO: 9 - DNA, 33 bp, artificial sequence; identical to SEQ ID NO: 7
  2. PCR Primer left

SEQ ID NO: 10 - DNA, 33 bp, artificial sequence
  2. PCR Primer right
ttatatgagctcttactcggccgtcggagatgg